(19) United States
(12) Patent Application Publication
(10) Pub. No.: US 2011/0223180 A1
(43) Pub. Date: Sep. 15, 2011

(54) METHODS AND COMPOSITIONS RELATED TO A MULTI-METHYLATION ASSAY TO PREDICT PATIENT OUTCOME

(76) Inventors: KENNETH D. ALDAPE, Bellaire, TX (US); Kristin Diefes, Houston, TX (US)

(21) Appl. No.: 13/044,934

(22) Filed: Mar. 10, 2011

Related U.S. Application Data
(60) Provisional application No. 61/312,976, filed on Mar. 11, 2010.

(51) Int. Cl. A61K 39/395; C12Q 1/68; C40B 30/00; A61K 48/00; A61P 35/00; A61N 5/00

(52) U.S. Cl. 424/174.1; 435/6.11; 506/7; 514/44 R; 600/1

(57) ABSTRACT

Methods and compositions for the prognosis and classification of cancer, especially brain tumor, are provided. For example, in certain aspects methods for cancer prognosis using methylation analysis of selected biomarkers are described.

The present application claims the priority benefit of U.S. provisional application No. 61/312,976, filed Mar. 11, 2010, the entire contents of which are incorporated herein by reference.

This invention was made with government support under NIH/NCI grants U24 CA126561 and U24 CA143882-01 and SPORE grant P50CA127001 awarded by the National Institute of Health. The government has certain rights in the invention.

### BACKGROUND OF THE INVENTION

1. Field of the Invention

The present invention relates generally to the fields of oncology, molecular biology, cell biology, and cancer. More particularly, it concerns cancer prognosis or treatment using molecular markers.

2. Description of Related Art

Cancer can be caused by the accumulation of both genetic and epigenetic alterations frequently leading to downstream changes in gene expression patterns. Epigenetic changes do not alter the DNA sequence, and therapeutics targeted at reversing epigenetic modifications hold the potential to reactivate expression of previously silenced genes, potentially altering the malignant phenotype. Furthermore, these epigenetic changes can be used as markers for detection of malignant cells in bodily fluids or solid samples. Epigenetic profiling technology has been promising to predict clinical outcomes and survival rates and to identify potential therapeutic targets and prognostic marker genes. Better understanding of the fundamental biology of epigenetic changes in cancer may not only improve prognostication but also offer new individualized therapeutic options.

However, despite many attempts to establish pre-treatment prognostic markers to understand the clinical biology of patients with cancer, validated clinical or biomarker epigenetic parameters are lacking in many aspects. Therefore, there remains a need to discover novel prognostic markers for cancer patients, especially brain cancer patients.

### SUMMARY OF THE INVENTION

Certain aspects of the invention are based, in part, on the discovery of a distinct subset of samples that displays a methylation phenotype having concerted hypermethylation and/or hypomethylation at a large number of loci during profiling of promoter DNA methylation alterations in glioblastoma (GBM) tumors. The methylation phenotype were found to be correlated with patients that are younger at the time of diagnosis and experience significantly improved outcome, adjusting for age and tumor grade.

Therefore, certain aspects of the present invention overcomes major deficiencies in the art by providing novel methods for determining whether a subject's cancer has a favorable methylation phenotype. In certain aspects for obtaining prognostic information, if the subject's cancer has the favorable methylation phenotype, the subject is more likely to exhibit a favorable prognosis. In other aspects, if the subject's cancer does not have the favorable methylation phenotype, the subject is less likely to exhibit a favorable prognosis.

To determine if a subject's cancer has a favorable methylation phenotype, there may be provided a method comprising determining methylation status of one or more of methylation markers in Table 1. In particular aspects, two or more of methylation markers in Table 1 may be determined. In certain aspects, a methylation status of two or more methylation markers with directionality of methylation status thereof specified in Table 1 may be indicative of such a favorable methylation phenotype.

In exemplary aspects, the methylation markers may include ANKRD43 (ankyrin repeat domain 43) gene, HFE (human hemochromatosis protein) gene, MAL (T cell differentiation protein MAL) gene, LGALS3 (galectin-3) gene, FAS-1 marker, FAS-2 marker, RHO-F (ras homolog gene family, member F) gene, WWTR1 (WW domain containing transcription regulator 1) gene, or DOCK5 (dedicator of cytokinesis 5) gene. The FAS-1 marker (also known as peFAScg983; position 41554652 of FAS gene) and FAS-2 marker (also known as psFAScg244; position 41554657 of FAS gene) involve the same gene FAS (also known as TNF receptor superfamily, member 6), but involve independent DNA sequences along that gene.

In a further aspect, there may be provided a method comprising determining whether a subject's cancer has a methylation status of one, two, or more of methylation markers with directionality of methylation status thereof specified in Table 1, such as a) hyper-methylation of ANKRD43 gene; b) hyper-methylation of HFE gene; c) hyper-methylation of MAL gene; d) hyper-methylation of LGALS3 gene; e) hyper-methylation of FAS-1 marker; f) hyper-methylation of FAS-2 marker; g) hyper-methylation of RHO-F gene; h) hyper-methylation of WWTR1 gene; and i) hypo-methylation of DOCK5 gene. In a preferable aspect, a methylation status of seven, eight, or all of a)-i) may be determined. In certain aspects, the existence of a methylation status of one, two, three, five, six, seven or more of the a) through i) may be indicative of a favorable methylation phenotype. Particularly, the existence of a methylation status of seven or more of the a) through i) may be indicative of a favorable methylation phenotype.

As used herein, “hypermethylation” or “hypomethylation” refers to a methylation level of a methylation biomarker in the subject's cancer as compared to a reference level representing the same methylation biomarker. In certain aspects, the reference level may be a reference level of methylation from non-cancerous tissue from the same subject. Alternatively, the reference level may be a reference level of methylation from a different subject or group of subjects. For example, the reference level of methylation may be a methylation level obtained from tissue of a subject or group of subjects without cancer, or a methylation level obtained from non-cancerous tissue of a subject or group of subjects with cancer. The reference level may be a single value or may be a range of values. The reference level of methylation can be determined using any method known to those of ordinary skill in the art. In some embodiments, the reference level is an average level of methylation determined from a cohort of subjects with cancer or without cancer. The reference level may also be depicted graphically as an area on a graph.

In a certain aspect, the subject is a human. The subject may have or be suspected to have cancer. The subject may be determined to have a cancer or be at risk for a cancer. The cancer related to the subject may be a cancer of brain, lung, liver, spleen, kidney, lymph node, small intestine, pancreas, blood cells, colon, stomach, breast, endometrium, prostate, testicle, ovary, skin, head and neck, esophagus, bone marrow or blood. For example, the cancer may be a primary brain tumor or a secondary brain tumor. In a particular aspect, the cancer may be glioma, more particularly, glioblastoma, or more particularly, secondary glioblastoma. In a further aspect, the cancer may be a recurrent cancer.

In certain aspects, the targets for methylation determination in cancer cells may be promoter regions containing G:C- and CpG-rich stretches of DNA, called ‘CpG islands.’ CpG islands are G:C and CpG-rich stretches of DNA in the genome, often located in the vicinity of genes, and generally unmethylated in normal somatic tissues. Therefore, aspects of the invention may comprise determining one or more non-coding regions of the methylation markers described above, particularly promoter regions.

In further aspects, there may be provided methods that comprise obtaining a sample of the subject. For assessing biomarker methylation, the sample may be serum, saliva, biopsy or needle aspirate. In a further aspect, the sample may be paraffin-embedded or frozen. In a particular aspect, the sample may be preserved, particularly, a formalin-fixed, paraffin-embedded (FFPE) sample.

The method may further comprise isolation nucleic acid of the subject's cancer. In particular aspects, the method may comprise assaying nucleic acids of the subject's cancer, in particular for one or more of the biomarkers described above.

The skilled artisan will understand that any methods known in the art for assessing methylation can be used in the present methods and compositions. The testing to assess methylation of the nucleic acids may include, but are not limited to, Southern blotting, single nucleotide primer extension (SNuPE), methylation-specific PCR (MSPCR), restriction landmark genomic scanning for methylation (RLGS-M), HpaII-tiny fragment enrichment by ligation-mediated PCR (HELP assay), CpG island microarray, ChIP-chip (chromatin immnuprecipitation-on-chip), ChIP-seq (chromatin immunoprecipitation-sequencing), methylated DNA immunoprecipitation (MeDIP), bisulfate sequencing, combined bisulfite restriction analysis (COBRA) or a microarray-based methylation profiling. For example, the microarray-based methylation profiling may be Infinium® methylation assay or GoldenGate® methylation assay.

In an alternative aspect, there may be provided methods that comprise analyzing a predetermined methylation profile. The predetermined methylation profile may be obtained from a lab, a service provider, or a technician.

In a further aspect, the method may comprise recording the methylation determination in a tangible medium. For example, such a tangible medium may be a computer-readable medium, such as a computer-readable disk, a solid state memory device, an optical storage device or the like, more specifically, a storage device such as a hard drive, a Compact Disk (CD) drive, a floppy disk drive, a tape drive, a random access memory (RAM), etc.

Based on the prognosis information, the methods may comprise reporting the methylation phenotype determination to the subject, a health care payer, a physician, an insurance agent, or an electronic system.

In certain aspects of the invention, the poor prognosis may indicate high risk of recurrence, poor survival, higher chance of cancer progress or metastasis, or a low response to or a poor clinical outcome after a conventional therapy such as surgery, chemotherapy and/or radiation therapy. In other aspects, the favorable prognosis may comprise low risk of recurrence, higher chance of survival, lower chance of cancer progress or metastasis, or a high response to or a favorable clinical outcome after a conventional therapy. In a particular aspect, the favorable prognosis may comprise a higher chance of survival as compared with a reference level. The poor or favorable prognosis may be determined as compared to a reference level. Such as a reference level may be obtained from an individual or a cohort group of subject, such as an mean or average level of survival.

Certain aspects of the methods also comprise methods for treating subjects that with a predetermined methylation status of one or more methylation biomarkers as described above. In further aspects, the methods may comprise prescribing or administering a treatment to the subject: for example, such a treatment would be a conventional therapy like surgery, chemotherapy and/or radiation therapy to the subject if favorable prognosis is identified, or an alternative treatment other than surgery, chemotherapy and radiation therapy to the subject if poor prognosis is identified.

In a further aspect, there may be provided a method of developing a treatment plan for a cancer patient comprising determining whether the patient's cancer has a favorable methylation phenotype and developing the treatment plan. In certain aspects, there may be methods comprising treating the subject with one or more conventional cancer treatments if the subject has the favorable methylation phenotype. The one or more conventional cancer treatments may comprise chemotherapy, radiation therapy, and/or surgery. In other aspects, there may be methods comprising treating the subject with one or more alternative cancer treatments if the subject does not have the favorable methylation phenotype. For example, the one or more alternative cancer treatments include, but are not limited to, angiogenesis inhibitor therapy, immunotherapy, gene therapy, hyperthermia, photodynamic therapy, and/or targeted cancer therapy.

In a still further aspect, there may also be provided a kit comprising a plurality of primers or probes specific for determining methylation status of one, two, three, five, six, seven or more methylation biomarkers in Table 1, such as ANKRD43 gene; HFE gene; MAL gene; LGALS3 gene; FAS-1 marker; FAS-2 marker; RHO-F gene; WWTR1 gene; and DOCK5 gene. In a particular aspect, the methylation status of at least two of the biomarkers may be determined by the kit.

In a further aspect, the kit may also comprise instructions to indicate that a subject has a favorable prognosis if a cancer sample from the subject has a favorable methylation phenotype as determined above; or to indicate that a subject has a poor prognosis if the sample does not have such a favorable methylation phenotype.

In other aspects, there may also be provided a tangible, computer-readable medium comprising a methylation profile of a subject, wherein the methylation profile comprises methylation status of one, two, three, five, six, seven or more methylation biomarkers in Table 1, such as ANKRD43 gene; HFE gene; MAL gene; LGALS3 gene; FAS-1 marker; FAS-2 marker; RHO-F gene; WWTR1 gene; and DOCK5 gene. In a particular aspect, the methylation profile may comprise a methylation status of at least two of the biomarkers.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan; however, these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. Methylation patterns in grade II, III and IV gliomas using MethyLight. FIG. 1A) Methylation profiling of gliomas shows an association of methylation marker status with tumor grade. Eight markers were tested for DNA methylation in 360 tumor samples. Each marker was coded as red if methylated and green if unmethylated. Markers are as follows: 1. DOCK5; 2. ANKRD43; 3. HFE; 4. MAL; 5. LGALS3; 6. FAS-1; 7. FAS-2; 8. RHOF; 9. WWTR1. WHO grade II tumors are shown on the upper left, WHO grade III tumors on the lower left and who grade IV tumors are shown on the right. One of these markers (DOCK5) is unmethylated in favorable survivors, while the remaining eight markers are hypermethylated in favorable survivors (methylation status of these markers in favorable survivors designated as being positive). It can be appreciated that the frequency of methylation of these markers differ according to tumor grade. FIG. 1B) Association of methylation marker status with patient outcome stratified by tumor grade. Cases with at least 7 of 9 positive markers are indicated by the dashed lines and remainder of cases are indicated by solid lines in each Kaplan-Meier survival curve. FIG. 1C) Stability of methylation status over time in glioma patients. Fifteen samples from newly diagnosed tumors were tested for methylation using 8 out of the 9 methylation markers (as indicated in the figure). Eight tumors were classified as favorable methylation panel (upper right panel), and seven tumors were classified as unfavorable methylation panel (lower left panel). Samples from a second procedure, ranging from 2-9 years after the initial resection, were also evaluated for the methylation status for favorable (upper right panel), as well as for the non-favorable cases (lower right panel).

### DESCRIPTION OF ILLUSTRATIVE EMBODIMENTS

Certain embodiments of the invention relate to determination of a genetic profile and use of the profile in cancer prognosis and personalized treatments. Certain aspects of the present invention are based, in part, on the identification of a genetic profile that are associated with clinical outcome and could therefore serve as a clinical test to predict outcome in cancer patients, especially glioma patients. In particular aspects, the genetic profile could be classified by a set of methylation biomarkers in Table 1, such as hypermethylated loci (e.g., ANKRD43, HFE, MAL, LGALS3, FAS-1, FAS-2, RHO-F, WWTR1) and/or one hypomethylated locus, DOCK5, which appear highly predictive and technically feasible to assay.

### I. DEFINITIONS

“Prognosis” refers to as a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognosis also includes prediction of favorable responses to cancer treatments, such as a conventional cancer therapy.

A favorable or poor prognosis may, for example, be assessed in terms of patient survival, likelihood of disease recurrence or disease metastasis. Patient survival, disease recurrence and metastasis may for example be assessed in relation to a defined time point, e.g. at a given number of years after a cancer treatment (e.g. surgery to remove one or more tumors) or after initial diagnosis. In one embodiment, a favorable or poor prognosis may be assessed in terms of overall survival or disease free survival.

By “subject” or “patient” is meant any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

### II. METHYLATION CHANGES

Currently, there are only crude clinical measurements that are largely based on patient status that are used to predict the clinical course of cancer patients, such as individuals stricken with glioblastoma (GBM). Lacking is a more tumor-based, biologic assessment that can be used to predict clinical outcomes in these patients and considered in the tailoring of more personalized therapeutic regimens.

Certain aspects of the invention identified a genetic profile with characteristic DNA methylation alterations, referred to as a favorable methylation phenotype that could be identified by two or more methylation biomarkers in Table 1, such as hypermethylated loci (e.g., ANKRD43, HFE, MAL, LGALS3, FAS-1, FAS-2, RHO-F, WWTR1) and/or one hypomethylated locus, DOCK5.

### III. METHYLATION BIOMARKERS

Although 70% to 80% of CpGs in human cells are normally methylated, cytosines within CpG islands are protected from methylation (Momparler, 2003). Even as CpG islands are typically unmethylated, the areas flanking the islands are methylated and act as barriers protecting against aberrant promoter methylation (Graff et al., 1997). In neoplasia, the barriers protecting the promoter CpG islands are commonly overridden with de novo methylation believed to begin at the distal ends of the island and then progressively spreading into the core. Therefore, cancer-associated hypermethylation is a dynamic process that may change with time, disease state, or treatment.

In certain aspects of the invention, methods and compositions are disclosed to use several methylation biomarkers for determining a subtype of cancer patients and for cancer prognosis and treatment optimizations. Specifically, a distinct subset of cancer samples displays concerted hypermethylation and/or hypomethylation at a large number of loci was identified as shown in Table 1, and was characterized as indicating the existence of a favorable methylation phenotype. In particular aspects, the existence of the favorable methylation phenotype may be identified by determination of methylation status of one, two, or more biomarkers in Table 1. In the left column of Table 1, the official gene symbols as provided by NCBI are used. If the gene symbols are entered into the “entrez gene” page on NCBI web site (at world wide web through.ncbi.nlm.nih.gov/sites/entrez?db=gene), with limits to homo sapiens, a unique gene ID will be identified.

TABLE 1
Methylation Markers

| | Directionality of Methylation status in |
|---|---|
| Gene symbol | favorable methylation phenotype |
| ACAA1 | Hypermethylated |
| ACAA2 | Hypermethylated |
| ACADS | Hypermethylated |
| ACTA1 | Hypermethylated |
| ACTC | Hypermethylated |
| ADAM12 | Hypermethylated |
| ADAM33 | Hypermethylated |
| ADCY8 | Hypermethylated |
| ADPRH | Hypermethylated |
| AGC1 | Hypermethylated |
| AKAP1 | Hypermethylated |
| ALDH1A3 | Hypermethylated |
| ALOX15B | Hypermethylated |
| AMID | Hypermethylated |
| AMIGO2 | Hypermethylated |
| AMMECR1 | Hypermethylated |
| ANKRD43 | Hypermethylated |
| ANXA2 | Hypermethylated |
| ARHGAP24 | Hypermethylated |
| ARTN | Hypermethylated |
| ASAHL | Hypermethylated |
| ATP5G2 | Hypermethylated |
| B3GNT5 | Hypermethylated |
| BCAT2 | Hypermethylated |
| BCL2 | Hypermethylated |
| BCORL1 | Hypermethylated |
| BEX2 | Hypermethylated |
| BZRP | Hypermethylated |
| C10orf35 | Hypomethylated |
| C11orf45 | Hypermethylated |
| C14orf50 | Hypermethylated |
| C19orf30 | Hypomethylated |
| C1orf187 | Hypermethylated |
| C21orf63 | Hypermethylated |
| C22orf8 | Hypermethylated |
| CABYR | Hypomethylated |
| CASK | Hypermethylated |
| CAV2 | Hypomethylated |
| CBR1 | Hypermethylated |
| CCL14 | Hypomethylated |
| CD58 | Hypermethylated |
| CDC14B | Hypermethylated |
| CDH3 | Hypermethylated |
| CENTD1 | Hypermethylated |
| CGI-38 | Hypermethylated |
| CHD5 | Hypermethylated |
| CHDH | Hypermethylated |
| CHFR | Hypomethylated |
| CHRNB1 | Hypermethylated |
| CHST8 | Hypermethylated |
| CNKSR2 | Hypermethylated |
| COL19A1 | Hypermethylated |
| COL21A1 | Hypermethylated |
| CRIP3 | Hypermethylated |
| CRYBA2 | Hypermethylated |
| CYP27A1 | Hypermethylated |
| D4ST1 | Hypermethylated |
| DAPK1 | Hypermethylated |
| DEDD2 | Hypermethylated |
| DFNB31 | Hypermethylated |
| DKFZp434N062 | Hypermethylated |
| DOCK5 | Hypomethylated |
| DOK1 | Hypermethylated |
| DOK5 | Hypermethylated |
| DSC2 | Hypermethylated |
| EBP | Hypermethylated |
| ECHDC2 | Hypermethylated |
| EDG3 | Hypermethylated |
| EFEMP1 | Hypermethylated |
| EFNB1 | Hypermethylated |
| EMP3 | Hypermethylated |
| EPHX2 | Hypermethylated |
| ERBB2 | Hypermethylated |
| ESAM | Hypermethylated |
| ESR2 | Hypermethylated |
| ESX1 | Hypermethylated |
| FAM11A | Hypermethylated |
| FAM58A | Hypermethylated |
| FAM70B | Hypermethylated |
| FAS | Hypermethylated |
| FES | Hypermethylated |
| FGF20 | Hypermethylated |
| FGFR3 | Hypermethylated |
| FGFRL1 | Hypermethylated |
| FKBP9 | Hypermethylated |
| FLJ12056 | Hypermethylated |
| FLJ20516 | Hypermethylated |
| FLJ20699 | Hypermethylated |
| FLJ23554 | Hypermethylated |
| FLJ33718 | Hypermethylated |
| FLJ45803 | Hypermethylated |
| FOXE1 | Hypermethylated |
| FRMD6 | Hypermethylated |
| FRZB | Hypermethylated |
| G6PD | Hypermethylated |
| GATA4 | Hypomethylated |
| GLOXD1 | Hypermethylated |
| GLT8D2 | Hypermethylated |
| GMPR | Hypermethylated |
| GNG13 | Hypermethylated |
| GNMT | Hypermethylated |
| GOLT1A | Hypermethylated |
| GPC4 | Hypermethylated |
| GPRC5A | Hypermethylated |
| GRASP | Hypermethylated |
| GSH2 | Hypermethylated |
| GUP1 | Hypermethylated |
| HAPLN3 | Hypermethylated |
| HCA112 | Hypermethylated |
| HCRT | Hypermethylated |
| HDAC3 | Hypermethylated |
| HFE | Hypermethylated |
| HIST1H4D | Hypermethylated |
| HMGB3 | Hypermethylated |
| HPCA | Hypermethylated |
| HPCAL4 | Hypermethylated |
| HSD11B2 | Hypermethylated |
| IGF2AS | Hypomethylated |
| INSIG1 | Hypermethylated |
| ITGA8 | Hypomethylated |
| KCNB2 | Hypomethylated |
| KCNH3 | Hypermethylated |
| KCNJ3 | Hypermethylated |
| KLC3 | Hypomethylated |
| KLF16 | Hypermethylated |
| KLK10 | Hypomethylated |
| LAMB1 | Hypomethylated |
| LENG9 | Hypermethylated |
| LGALS3 | Hypermethylated |
| LLGL2 | Hypermethylated |
| LOXL4 | Hypermethylated |
| LRAT | Hypermethylated |
| LRRC56 | Hypermethylated |
| LRRFIP1 | Hypermethylated |
| LTBP1 | Hypermethylated |
| MAL | Hypermethylated |
| MASK | Hypermethylated |
| MCF2L2 | Hypermethylated |
| MDK | Hypermethylated |
| MED12 | Hypermethylated |
| MEGF10 | Hypermethylated |
| MGC35308 | Hypermethylated |
| MGC9850 | Hypermethylated |
| MGST2 | Hypermethylated |
| MMP14 | Hypermethylated |
| MOSC1 | Hypermethylated |
| MOSC2 | Hypermethylated |
| MRCL3 | Hypermethylated |
| MT1E | Hypermethylated |
| MT1F | Hypermethylated |
| MT1X | Hypermethylated |
| MTCP1 | Hypermethylated |
| MTMR1 | Hypermethylated |
| MYRIP | Hypermethylated |
| NPAL2 | Hypomethylated |
| NUDT14 | Hypermethylated |
| NUDT16 | Hypermethylated |
| OCRL | Hypermethylated |
| OSRF | Hypomethylated |
| OTUB1 | Hypomethylated |
| PAH | Hypermethylated |
| PDCD6IP | Hypomethylated |
| PDE8A | Hypermethylated |
| PERP | Hypermethylated |
| PGK1 | Hypermethylated |
| PGRMC1 | Hypermethylated |
| PHKA1 | Hypermethylated |
| PIR | Hypermethylated |
| PLA2G3 | Hypermethylated |
| PODN | Hypermethylated |
| PORCN | Hypermethylated |
| PRODH | Hypermethylated |
| PRPS2 | Hypermethylated |
| PSMD10 | Hypermethylated |
| PTGDR | Hypomethylated |
| PTMS | Hypomethylated |
| PTPRN | Hypomethylated |
| PVT1 | Hypermethylated |
| RAB11FIP5 | Hypermethylated |
| RAB27B | Hypermethylated |
| RAB32 | Hypermethylated |
| RAB33A | Hypermethylated |
| RAB34 | Hypermethylated |
| RAB3D | Hypermethylated |
| RAP1GA1 | Hypermethylated |
| RASGEF1A | Hypermethylated |
| RBP1 | Hypermethylated |
| RHOF | Hypermethylated |
| RILP | Hypermethylated |
| RNF190 | Hypermethylated |
| RNF39 | Hypermethylated |
| RPP25 | Hypermethylated |
| SERPINI1 | Hypomethylated |
| SFRP4 | Hypermethylated |
| SH3BP4 | Hypermethylated |
| SLITL2 | Hypermethylated |
| SMOC2 | Hypomethylated |
| SNF1LK | Hypermethylated |
| SOCS2 | Hypomethylated |
| SPATS1 | Hypermethylated |
| STEAP3 | Hypermethylated |
| STK6 | Hypomethylated |
| SULT1A3 | Hypermethylated |
| TAT | Hypomethylated |
| TBL2 | Hypermethylated |
| TCEAL3 | Hypermethylated |
| TETRAN | Hypermethylated |
| THBS1 | Hypermethylated |
| TIMP1 | Hypermethylated |
| TMEM106A | Hypermethylated |
| TMEM63A | Hypermethylated |
| TNK2 | Hypomethylated |
| TOM1L1 | Hypermethylated |
| TP73 | Hypermethylated |
| TPPP | Hypermethylated |
| TRIM25 | Hypermethylated |
| TRIM59 | Hypomethylated |
| TRIP6 | Hypermethylated |
| TSSK3 | Hypermethylated |
| TTC22 | Hypomethylated |
| TUBA1 | Hypermethylated |
| TUBA6 | Hypermethylated |
| TUBA8 | Hypermethylated |
| TWIST1 | Hypomethylated |
| UCP2 | Hypermethylated |
| UNC5A | Hypermethylated |
| VCL | Hypermethylated |
| VIL2 | Hypermethylated |
| VILL | Hypermethylated |
| WDR44 | Hypermethylated |
| WNT6 | Hypomethylated |
| WWTR1 | Hypermethylated |
| ZMYND10 | Hypermethylated |
| ZNF206 | Hypomethylated |
| ZNF342 | Hypermethylated |

For example, when using preserved samples, such as FFPE samples, methylation status such as hypermethylated loci (e.g., ANKRD43, HFE, MAL, LGALS3, FAS-1, FAS-2, RHO-F, WWTR1) and/or one hypomethylated locus, DOCK5 may be used for methylation phenotype evaluation. The presence of one, two, three, four, five, six, seven, eight, or nine of the biomarker methylation status may indicate the presence of the favorable methylation phenotype.

In some embodiments, the presence or absence or quantity of methylation of the chromosomal DNA within a DNA region or portion thereof (e.g., at least one cytosine) of the one or more methylation biomarkers is detected. Portions of the DNA regions described herein may comprise at least one potential methylation site (i.e., a cytosine) and can generally comprise 2, 3, 4, 5, 10, or more potential methylation sites. In some embodiments, the methylation status of one or more cytosines within a methylation biomarker is detected.

In some embodiments, the methylation of at least one cytosine in more than one DNA region (or portion thereof) may be detected. In particular embodiments, the methylation status of 1, 2, 3, 4, 5, 6, 7, 8, or 9 of the methylation marker DNA regions may be determined.

In some embodiments of the invention, the methylation of a DNA region or portion thereof is determined and then normalized (e.g., compared) to the methylation of a control locus. Typically the control locus will have a known, relatively constant, methylation status. For example, the control sequence can be previously determined to have no, some or a high amount of methylation, thereby providing a relative constant value to control for error in detection methods, etc., unrelated to the presence or absence of cancer. In some embodiments, the control locus is endogenous, i.e., is part of the genome of the individual sampled. For example, in mammalian cells, the testes-specific histone 2B gene (hTH2B in human) gene is known to be methylated in all somatic tissues except testes. Alternatively, the control locus can be an exogenous locus, i.e., a DNA sequence spiked into the sample in a known quantity and having a known methylation status. Such exogenous sequences can be methylated in vitro, if desired, using a DNA methylase.

A DNA region comprises a nucleic acid including one or more methylation sites of interest (e.g., a cytosine, a “microarray feature,” or an amplicon amplified from select primers) and flanking nucleic acid sequences (i. e., “wingspan”) of up to 4 kilobases (kb) in either or both of the 3′ or 5′ direction from the amplicon. This range corresponds to the lengths of DNA fragments obtained by randomly fragmenting the DNA before screening for differential methylation between DNA in two or more samples (e.g., carrying out methods used to initially identify differentially methylated sequences as described in the Examples, below). In some embodiments, the wingspan of the one or more DNA regions is about 0.5 kb, 0.75 kb, 1.0 kb, 1.5 kb, 2.0 kb, 2.5 kb, 3.0 kb, 3.5 kb or 4.0 kb in both 3′ and 5′ directions relative to the sequence represented by the microarray feature.

The methylation sites in a DNA region can reside in non-coding transcriptional control sequences (e.g., promoters, enhancers, etc.) or in coding sequences, including introns and exons of the identified genes. In some embodiments, the methods comprise detecting the methylation status in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional state site through to the transcriptional start site) of one or more of the genes identified herein.

A. ANKRD43 (Ankyrin Repeat Domain 43) Gene;

Homo sapiens gene ANKRD43, encoding ankyrin repeat domain 43, maps on chromosome 5, at 5q31.1 according to Entrez Gene. In AceView developed at the National Center for Biotechnology Information (NCBI), it covers 3.48 kb, from 132176914 to 132180393 (NCBI 36, March 2006), on the direct strand. The gene is also known as ANKRD43, LOC134548. It has been described as ankyrin repeat domain 43. The sequence of this gene is defined by 59 GenBank accessions from 52 cDNA clones, some from hypothalamus (seen 12 times), kidney (7), hippocampus (6), prostate (5), whole brain (5), multiple sclerosis lesions (4), small intestine (4) and 14 other tissues. This gene contains ankyrin domain. No phenotype has yet been reported to the inventors' knowledge: this gene's in vivo function is yet unknown.

B. HFE (Human Hemochromatosis Protein) Gene;

The HFE gene is found in region 21.3 on the short (p) arm of human chromosome 6. The HFE gene's seven coding regions (exons) are scattered over about 10,000 base pairs of genomic DNA. Exons translated into the HFE protein are interspersed with segments of noncoding DNA (introns). After transcription, introns are spliced out and exons are pieced together to form an mRNA transcript about 2700 bp long. The mRNA is then translated into the 348-amino acid sequence of the hereditary hemochromatosis protein (mRNA identifier number: NM_{—}000410; protein identifier number: NP_{—}000401). Mutations in the HFE gene can result in hereditary hemochromatosis (HH).

The HFE protein is a transmembrane protein expressed in intestinal and liver cells; it works in conjunction with another small protein called beta-2-microglobulin to regulate iron uptake. Although homologous to other major histocompatibility complex (MHC) class I proteins that present antigens to killer T cells, the HFE protein appears to have no immunological function. Instead, it regulates iron concentration through different mechanisms in different cell types. In some cells it decreases iron concentration while in others it increases it (Davies, 2004).

HFE protein consists of extracellular alpha-1 and alpha-2 domains that sit on top of the immunoglobulin-like alpha-3 domain, which spans the cell membrane and binds a separate protein called beta-2-microglobulin. The alpha-1 and apha-2 domains interact with the transferrin receptor, another transmembrane protein that plays a very important role in iron uptake and regulation.

C. MAL Gene

The MAL gene is also known as a gene encoding T-cell differentiation protein MAL, T-lymphocyte maturation-associated protein or myelin and lymphocyte protein. The protein encoded by this gene is a highly hydrophobic integral membrane protein belonging to the MAL family of proteolipids. The protein has been localized to the endoplasmic reticulum of T-cells and is a candidate linker protein in T-cell signal transduction. In addition, this proteolipid is localized in compact myelin of cells in the nervous system and has been implicated in myelin biogenesis and/or function. The protein plays a role in the formation, stabilization and maintenance of glycosphingolipid-enriched membrane microdomains. Alternative splicing produces four transcript variants which vary from each other by the presence or absence of alternatively spliced exons 2 and 3.

MAL has a promoter CpG island of ˜1,500 bp that contains 116 CpG dinucleotides and extends into the first intron.

D. LGALS3 (Galectin-3) Gene

Galectin-3 is a protein that in humans is encoded by the LGALS3 gene. In melanocytic cells LGALS3 gene expression may be regulated by MITF. Galectin is a type of lectin which binds beta-galactoside. Galectins are widely distributed in animals with a wide variety of functions, including inhibition of chronic inflammations, GVHD, and allergic reactions. Galectin-3 is a polyllactosamine binding animal lectin, shown to be involved in tumor progression and metastasis.

E. Fas Receptor Genes (FAS-1 Marker and FAS-2 Marker)

The Fas receptor (FasR) is the most intensely studied death receptor. Its aliases include CD95, Apo-1, and tumor necrosis factor receptor superfamily, member 6 (TNFRSf6). The gene is situated on chromosome 10 in humans and 19 in mice. FAS orthologs have also been identified in most mammals for which complete genome data are available.

The protein encoded by this gene is a member of the TNF-receptor superfamily. This receptor contains a death domain. It has been shown to play a central role in the physiological regulation of programmed cell death, and has been implicated in the pathogenesis of various malignancies and diseases of the immune system. The interaction of this receptor with its ligand allows the formation of a death-inducing signaling complex that includes Fas-associated death domain protein (FADD), caspase 8, and caspase 10. The autoproteolytic processing of the caspases in the complex triggers a downstream caspase cascade, and leads to apoptosis. This receptor has been also shown to activate NF-kappaB, MAPK3/ERK1, and MAPK8/INK, and is found to be involved in transducing the proliferating signals in normal diploid fibroblast and T cells. At least eight alternatively spliced transcript variants have been described, some of which are candidates for nonsense-mediated decay (NMD). The isoforms lacking the transmembrane domain may negatively regulate the apoptosis mediated by the full length isoform.

The human Fas gene contains a 650 bp CpG island spanning the 50 flanking region of the gene, suggesting that CpG methylation could be responsible for downregulating Fas expression. The first intron, which contains a p53 responsive element, is also a region demonstrating high density of CpG sites.

F. RHO-F (Ras Homolog Gene Family, Member F) Gene

RHO-F gene encodes a plasma membrane-associated small GTPase which cycles between an active GTP-bound and an inactive GDP-bound state. This small GTPase causes the formation of thin, actin-rich surface projections called filopodia. RHO-F protein functions cooperatively with CDC42 and Rac to generate additional structures, increasing the diversity of actin-based morphology.

G. WWTR1 (WW Domain Containing Transcription Regulator 1)

Wwtr1 [WW-domain containing transcription regulator 1, also referred to as Taz (transcriptional coactivator with PDZ-binding motif)] is highly expressed in the kidney, heart, lung, liver, testis, and placenta. Wwtr1 is a widely expressed 14-3-3-binding protein that regulates the activity of several transcription factors involved in development and disease. Wwtr1 binds via a single WW domain to L/PPXY motifs in target transcription factors. Although Wwtr1 interacts with different transcription factors, little is known about the physiological role of Wwtr1 in vivo.

### IV. METHYLATION PHENOTYPE DETERMINATION

In certain aspects, this invention entails determining methylation information of one or more methylation biomarkers in a sample of cells from a subject with cancer. The methylation information may be obtained by testing cancer samples by a lab, a technician, a device, or a clinician or may be determined by any method known in the art.

A. Determining Methylation

Any method for detecting DNA methylation can be used in the methods of the present invention.

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Patent Publication No. 2004/0132048. Alternatively, the method can encompass amplifying intact DNA after restriction enzyme digestion, thereby only amplifying DNA that was not cleaved by the restriction enzyme in the area amplified. See, e.g., U.S. patent application Ser. Nos. 10/971,986; 11/071,013; and 10/971,339. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In some embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. patent application Ser. No. 10/971,986.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., Gibson et al. (1996); DeGraves et al. (2003); Deiman et al. (2002). Amplifications may be monitored in “real time.”

Additional methods for detecting DNA methylation can involve genomic sequencing before and after treatment of the DNA with bisulfite. See, e.g., Frommer et al. (1992). When sodium bisulfite is contacted to DNA, unmethylated cytosine is converted to uracil, while methylated cytosine is not modified.

In some embodiments, restriction enzyme digestion of PCR products amplified from bisulfite-converted DNA is used to detect DNA methylation. See, e.g., Sadri & Hornsby (1996); Xiong & Laird (1997).

In some embodiments, a MethyLight assay is used alone or in combination with other methods to detect DNA methylation (see, Eads et al., 1999). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably-labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Ms-SNuPE (Methylation-sensitive Single Nucleotide Primer Extension) reaction is used alone or in combination with other methods to detect DNA methylation (see, Gonzalgo & Jones, 1997). The Ms-SNuPE technique is a quantitative method for assessing methylation differences at specific CpG sites based on bisulfite treatment of DNA, followed by single-nucleotide primer extension (Gonzalgo & Jones, supra). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated versus unmethylated DNA. See, Herman et al. (1996); U.S. Pat. No. 5,786,146.

Additional methylation detection methods include, but are not limited to, methylated CpG island amplification (see, Toyota et al., 1999) and those described in, e.g., U.S. Patent Publication 2005/0069879; Rein et al. (1998); Olek et al. (1997); and PCT Publication No. WO 00/70090.

B. Determining Gene and Protein Expression

It is well known that methylation of genomic DNA can affect expression (transcription and/or translation) of nearby gene sequences. Therefore, in some embodiments, the methods include the step of correlating the methylation status of at least one cytosine in a DNA region of the methylation biomarkers as described above with the expression of nearby coding sequences. For example, expression of gene sequences within about 1.0 kb, 1.5 kb, 2.0 kb, 2.5 kb, 3.0 kb, 3.5 kb or 4.0 kb in either the 3′ or 5′ direction from the cytosine of interest in the DNA region can be detected. In some embodiments, the gene or protein expression of one or more methylation biomarkers is compared to a control, for example, the methylation status in the DNA region and/or the expression of a nearby gene sequence from a sample from an individual known to be negative for cancer or known to be positive for cancer, or to an expression level that distinguishes between cancer and noncancer states. Such methods, like the methods of detecting methylation described herein, are useful in providing diagnosis, prognosis, etc., of cancer. Methods for measuring transcription and/or translation of a particular gene sequence are well known in the art. See, for example, Ausubel, Current Protocols in Molecular Biology, 1987-2006, John Wiley & Sons; and Sambrook and Russell, Molecular Cloning: A Laboratory Manual, 3rd Edition, 2000.

In some embodiments, the methods further comprise the step of correlating the methylation status and expression of one or more of the gene regions of the one or more methylation biomarkers as describe above.

Certain aspects of the present invention thus provides for detection of gene (e.g. RNA) and/or protein expression to detect cancer, particularly brain cancer. RNA or protein expression from the genomic regions described herein can be compared to a reference level or otherwise normal expression (e.g., expression for normal, non-cancerous tissue) to detect cancer, particularly brain cancer. In some embodiments, methylation biomarker expression is detected and compared to a reference value or otherwise normal expression (i.e., expression for normal, non-cancerous tissue) of methylation biomarker.

Any method of detecting RNA or protein expression can be used in the methods of certain aspects of the invention. In some embodiments, the presence of cancer is evaluated by determining the level of expression of mRNA encoding a protein of interest. Methods of evaluating RNA expression of a particular gene are well known to those of skill in the art, and include, inter alia, hybridization and amplification based assays.

Methods of detecting and/or quantifying the level of gene transcripts of interest (mRNA or cDNA made therefrom) using nucleic acid hybridization techniques are known to those of skill in the art. For example, one method for evaluating the presence, absence, or quantity of polynucleotides involves a northern blot. Gene expression levels can also be analyzed by techniques known in the art, e.g., dot blotting, in situ hybridization, RNase protection, probing DNA microchip arrays, and the like.

In another embodiment, amplification-based assays are used to measure the expression level of a gene of interest. In such an assay, the nucleic acid sequences act as a template in an amplification reaction (e.g., Polymerase Chain Reaction, or PCR). In a quantitative amplification, the amount of amplification product will be proportional to the amount of template in the original sample (e.g., can from a reverse transcription reaction of the target RNA). Comparison to appropriate controls provides a measure of the level of expression of the gene of interest in the sample. Methods of quantitative amplification are well known to those of skill in the art. Detailed protocols for quantitative PCR are provided, e.g., in Innis et al. (1990). The nucleic acid sequences provided herein are sufficient to enable one of skill to select primers to amplify any portion of the gene and/or encoded RNA.

In one non-limiting embodiment, a TaqMan™ based assay is used to quantify the cancer-associated polynucleotides. TaqMan™ based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan™ probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

Other suitable amplification methods include, but are not limited to, ligase chain reaction (LCR) (see, Wu and Wallace, 1989; Landegren et al., 1988; and Barringer et al., 1990; transcription amplification (Kwoh et al., 1989), self-sustained sequence replication (Guatelli et al., 1990), dot PCR, and linker adapter PCR, etc.

Polypeptides encoded by the genes described herein can be detected and/or quantified by any methods known to those of skill in the art from samples as described herein. In some embodiments, antibodies can also be used to detect polypeptides encoded by the genes described herein. Antibodies to these polypeptides can be produced using well known techniques (see, e.g., Harlow & Lane, 1988 and Harlow & Lane, 1999; Coligan, 1991; Goding, 1986; and Kohler & Milstein, 1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., 1989; Ward et al., 1989).

Once specific antibodies are available, binding interactions with the proteins of interest can be detected by a variety of immunoassay methods. For a review of immunological and immunoassay procedures, see Basic and Clinical Immunology (1991). Moreover, the immunoassays of certain aspects of the present invention can be performed in any of several configurations, which are reviewed extensively in Enzyme Immunoassay (1980); and Harlow & Lane, supra).

Immunoassays also often use a labeling agent to specifically bind to and label the complex formed by the antibody and antigen. The labeling agent may itself be one of the moieties comprising the antibody/antigen complex. Thus, the labeling agent may be a labeled polypeptide or a labeled antibody that binds the protein of interest. Alternatively, the labeling agent may be a third moiety, such as a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the labeling agent. These proteins exhibit a strong non-immunogenic reactivity with immunoglobulin constant regions from a variety of species (see, e.g., Kronval et al., 1973; Akerstrom et al., 1985). The labeling agent can be modified with a detectable moiety, such as biotin, to which another molecule can specifically bind, such as streptavidin. A variety of detectable moieties are well known to those skilled in the art.

Commonly used assays include noncompetitive assays, e.g., sandwich assays, and competitive assays. In competitive assays, the amount of polypeptide present in the sample is measured indirectly by measuring the amount of a known, added (exogenous) polypeptide of interest displaced (competed away) from an antibody that binds by the unknown polypeptide present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers. The released chemicals are then detected according to standard techniques (see Monroe et al., 1986).

### V. CANCER DETECTION

The present markers and methods can be used in the diagnosis, prognosis, classification, prediction of disease risk, detection of recurrence of disease, and selection of treatment of cancer, in particular, brain cancer. Any stage of progression can be detected, such as primary, metastatic, and recurrent cancer. Information regarding numerous types of cancer can be found, e.g., from the American Cancer Society (available on the worldwide web at cancer.org), or from, e.g., Harrison's Principles of Internal Medicine, (2005).

Certain aspects of the present invention provide methods for cancer prognosis, such as estimating the likelihood of a mammal developing cancer, classifying cancer stages, and monitoring the efficacy of anti-cancer treatment in a mammal with cancer. Such methods are based on the discovery that cancer cells differentially methylate DNA sequences at the methylation biomarker of certain aspects of the invention. Accordingly, by determining whether or not a cell contains a particular methylation profile including methylated DNA sequences in the DNA regions of one or more methylation biomarkers as described herein, preferably at least two of them, it is possible to determine whether or not the cancer has a favorable or poor prognosis. Similarly, as described herein, quantification of methylation biomarker levels in cancerous tissues may be used for cancer prognosis.

In numerous embodiments of the present invention, the presence of methylated nucleotides in the methylation biomarker DNA regions of certain aspects of the invention is detected in a biological sample, thereby detecting the presence or absence of cancerous cells in the biological sample. In some embodiments, the biological sample comprises a tissue sample from a tissue suspected of containing cancerous cells. Human genomic DNA samples can be obtained by any means known in the art. In cases where a particular phenotype or disease is to be detected, DNA samples should be prepared from a tissue of interest, or as appropriate, from cerebral spinal fluid. For example, DNA can be prepared from biopsy tissue to detect the methylation state of a particular locus associated with cancer.

The nucleic acid-containing specimen used for detection of methylated loci (see, e.g., Ausubel et al., Current Protocols in Molecular Biology (1995 supplement)) may be from any source and may be extracted by a variety of techniques such as those described by Ausubel et al. (1995) or Sambrook et al. (2001). Exemplary tissues include, e.g., brain tissue. As appropriate, the tissue or cells can be obtained by any method known in the art including by surgery. In other embodiments, a tissue sample known to contain cancerous cells, e.g., from a tumor, will be analyzed for the presence or quantity of methylation at one or more of the methylation biomarkers as described above to determine information about the cancer, particularly brain cancer, e.g., the efficacy of certain treatments, the survival expectancy of the individual, etc. In some embodiments, the methods may be used in conjunction with additional prognostic or diagnostic methods, e.g., detection of other cancer markers, etc.

The methods of certain aspects of the invention can be used to evaluate individuals known or suspected to have cancer, particularly brain cancer, or as a routine clinical test, e.g., in an individual not necessarily suspected to have cancer. Further diagnostic assays can be performed to confirm the status of cancer in the individual.

Further, the present methods may be used to assess the efficacy of a course of treatment. For example, the efficacy of an anti-cancer treatment can be assessed by monitoring DNA methylation of the marker sequences described herein over time in a mammal having cancer, particularly brain cancer. For example, a reduction or absence of methylation in any of the methylation biomarkers as described above in a biological sample taken from a mammal following a treatment, compared to a level in a sample taken from the mammal before, or earlier in, the treatment, indicates efficacious treatment.

The methods detecting cancer, particularly brain cancer, can comprise the detection of one or more other cancer-associated polynucleotide or polypeptides sequences. Accordingly, detection of methylation of any one or more of the methylation biomarkers as described above can be used either alone, or in combination with other markers, for the diagnosis or prognosis of cancer.

The methods of certain aspects of the present invention can be used to determine the optimal course of treatment in a mammal with cancer. For example, the presence of methylated DNA within any of the methylation biomarkers as described above or an increased quantity of methylation within any of the methylation biomarkers can indicate a reduced survival expectancy of a mammal with cancer, particularly brain cancer, thereby indicating a more aggressive treatment for the mammal. In addition, a correlation can be readily established between the presence, absence or quantity of methylation at a methylation biomarkers, as described herein, and the relative efficacy of one or another anti-cancer agent. Such analyses can be performed, e.g., retrospectively, i.e., by detecting methylation in one or more of the methylation biomarkers in samples taken previously from mammals that have subsequently undergone one or more types of anti-cancer therapy, and correlating the known efficacy of the treatment with the presence, absence or levels of methylation of one or more of the methylation biomarkers as described above.

In making a diagnosis, prognosis, risk assessment, classification, detection of recurrence or selection of therapy based on the presence or absence of methylation in at least one of the methylation biomarkers, the quantity of methylation may be compared to a threshold value that distinguishes between one diagnosis, prognosis, risk assessment, classification, etc., and another. For example, a threshold value can represent the degree of methylation found at a particular DNA region that adequately distinguishes between cancer samples and normal biopsy samples with a desired level of sensitivity and specificity. It is understood that a threshold value will likely vary depending on the assays used to measure methylation, but it is also understood that it is a relatively simple matter to determine a threshold value or range by measuring methylation of a DNA sequence in brain and normal samples using the particular desired assay and then determining a value that distinguishes at least a majority of the cancer samples from a majority of non-cancer samples. If methylation of two or more DNA regions is detected, two or more different threshold values (one for each DNA region) will often, but not always, be used.

In some embodiments, the methods comprise recording a diagnosis, prognosis, risk assessment or classification, based on the methylation status determined from an individual. Any type of recordation is contemplated, including electronic recordation, e.g., by a computer.

### VI. CANCER

Certain embodiments of the present invention provide for determination of methylation status in a subject's cancer. The methylation information may be used for cancan prognosis, assessment, classification and/or treatment. Cancers which may be examined by a method described herein may include, but are not limited to, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullar carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonic carcinoma, squamous cell carcinoma, base cell carcinoma, fibrosarcoma, myxoma, myxosarcoma, liposarcoma, chondrosarcoma, osteogenic sarcoma, leukemia, and the metastatic lesions secondary to these primary tumors.

A. Brain Cancer

In a particular aspect, the brain cancer may be assessed for its methylation profile by using the methods and compositions of the invention.

There are two types of brain cancer: primary brain tumors that originate in the brain and metastatic (secondary) brain tumors that originate from cancer cells that have migrated from other parts of the body.

Primary brain tumors rarely spreads beyond the central nervous system, and death results from uncontrolled tumor growth within the limited space of the skull. Metastatic brain cancer indicates advanced disease and has a poor prognosis.

Primary brain tumors can be cancerous or noncancerous. Both types take up space in the brain and may cause serious symptoms (e.g., vision or hearing loss) and complications (e.g., stroke).

All cancerous brain tumors are life threatening (malignant) because they have an aggressive and invasive nature. A noncancerous primary brain tumor is life threatening when it compromises vital structures (e.g., an artery). In the United States, the annual incidence of brain cancer generally is 15-20 cases per 100,000 people. Brain cancer is the leading cause of cancer-related death in patients younger than age 35.

Primary brain tumors account for 50% of intracranial tumors and secondary brain cancer accounts for the remaining cases. Approximately 17,000 people in the United States are diagnosed with primary cancer each year and nearly 13,000 die of the disease. The annual incidence of primary brain cancer in children is about 3 per 100,000.

Secondary brain cancer occurs in 20-30% of patients with metastatic disease and incidence increases with age. In the United States, about 100,000 cases of secondary brain cancer are diagnosed each year.

B. Glioma

More particularly, there may be provided methods and compositions for determining methylation status of a glioma patient.

Human gliomas present as heterogeneous disease, primarily defined by the cell of origin. Astrocytomas, derived from astrocytes, are the most abundant human gliomas, whereas oligodendrogliomas (derived from oligodendrocytes), ependymomas (derived from ependymal cells) and mixtures of glial cell types comprise a minority of diagnosed gliomas (Adamson et al., 2009). However, the identification of tumorigenic, stem-cell like precursor cells in advanced stage gliomas suggests that human gliomas may have a neural stem cell origin (Canoll and Goldman, 2008; Dirks, 2006; Galli et al., 2004).

Gliomas are subdivided by the World Health Organization (WHO) by histological grade, which is an indication of differentiation status, malignant potential, response to treatment and survival. Glioblastoma (GBM), also described as Grade IV glioma, accounts for more than 50% of all gliomas (Adamson et al., 2009). Patients with GBM have an overall median survival time of only 15 months (Brander et al., 2001; Martinez et al., 2009b; Parsons et al., 2008). Most GBMs are diagnosed as de novo, or primary tumors. A subset of ˜5% of GBM tumors, termed secondary GBM, progress from low grade tumors, are seen in younger patients, are more prevalent in women, and exhibit longer survival times (reviewed in (Adamson et al., 2009; Furnari et al., 2007).

There is currently great interest in characterizing and compiling the genome and transcriptome changes in human GBM tumors to identify aberrantly functioning molecular pathways and tumor subtypes. The Cancer Genome Atlas (TCGA) pilot project identified genetic changes of primary DNA sequence and copy number, DNA methylation, gene expression and patient clinical information for a set of GBM tumors (The Cancer Genome Atlas Research Network, 2008). TCGA also reaffirmed genetic alterations in TP53, PTEN, EGFR and RB1 in GBM patients, along with the novel identification of NF1, ERBB2, PIK3R1, and PIK3CA mutations (The Cancer Genome Atlas Research Network, 2008). Recent DNA sequencing analyses of primary GBM tumors using a more comprehensive approach (Parsons et al., 2008) also identified novel somatic mutations in isocitrate dehydrogenase 1 (IDH1) that occur in 12% of all GBM patients. IDH1 mutations have only been detected at the arginine residue in codon 132, with the most common change being the R132H mutation (Parsons et al., 2008; Yan et al., 2009), which results in a novel gain of enzyme function in directly catalyzing α-ketoglutarate to R(−)-2-hydroxyglutarate (Dang et al., 2009). IDH1 mutations are enriched in secondary GBM cases, younger individuals and coincident with increased patient survival (Balss et al., 2008; Hartmann et al., 2009; Yan et al., 2009). Higher IDH1 mutation rates are seen in grade II and III astrocytomas and oligodendrogliomas (Balss et al., 2008; Bleeker et al., 2009; Hartmann et al., 2009; Yan et al., 2009), suggesting that IDH1 mutations generally occur in the progressive form of glioma, rather than in de novo GBM. Mutations in the related IDH2 gene are of lower frequency and non-overlapping with tumors containing IDH1 mutations (Hartmann et al., 2009; Yan et al., 2009).

### VII. KITS

Certain aspects of this invention also provide kits for the detection and/or quantification of the methylation biomarkers, or expression or methylation thereof using the methods described herein.

For kits for detection of methylation, the kits can comprise at least one polynucleotide that hybridizes to at least one of the methylation biomarker sequences and at least one reagent for detection of gene methylation. Reagents for detection of methylation include, e.g., sodium bisulfate, polynucleotides designed to hybridize to sequence that is the product of a marker sequence if the marker sequence is not methylated (e.g., containing at least one C→U conversion), and/or a methylation-sensitive or methylation-dependent restriction enzyme. The kits can provide solid supports in the form of an assay apparatus that is adapted to use in the assay. In a particular aspect, kits for the methods of certain aspects of the present invention can include, e.g., one or more of methylation-dependent restriction enzymes, methylation-sensitive restriction enzymes, amplification (e.g., PCR) reagents, probes and/or primers.

The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In a certain aspect, these kits may comprise a plurality of agents for assessing the methylation of a plurality of methylation biomarkers, for example, one, two, three, four, five, six, seven or more of the methylation biomarkers as described above, wherein the kit is housed in a container. The kits may further comprise instructions for using the kit for assessing methylation, means for converting the methylation data into methylation values and/or means for analyzing the methylation data or values to generate prognosis. The agents in the kit for measuring biomarker methylation may comprise a plurality of probes and/or primers for methylation-sensitive extension or amplification of the biomarkers. In another embodiment, the agents in the kit for measuring biomarker methylation may comprise an array of polynucleotides complementary to the nucleic acid sequence of the biomarkers of the invention. Possible means for converting the methylation data into methylation values and for analyzing the methylation values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Prognostic Significance of Methylation Markers by a Multi-Methylation Assay

A pool of about 200 markers in Table 1 has been found in the present invention that biologically define a favorable methylation phenotype. Any subset of the ˜200 markers, as long as there were enough of them to show concordant hypermethylation (or hypomethylation), could be used to demonstrate this phenotype.

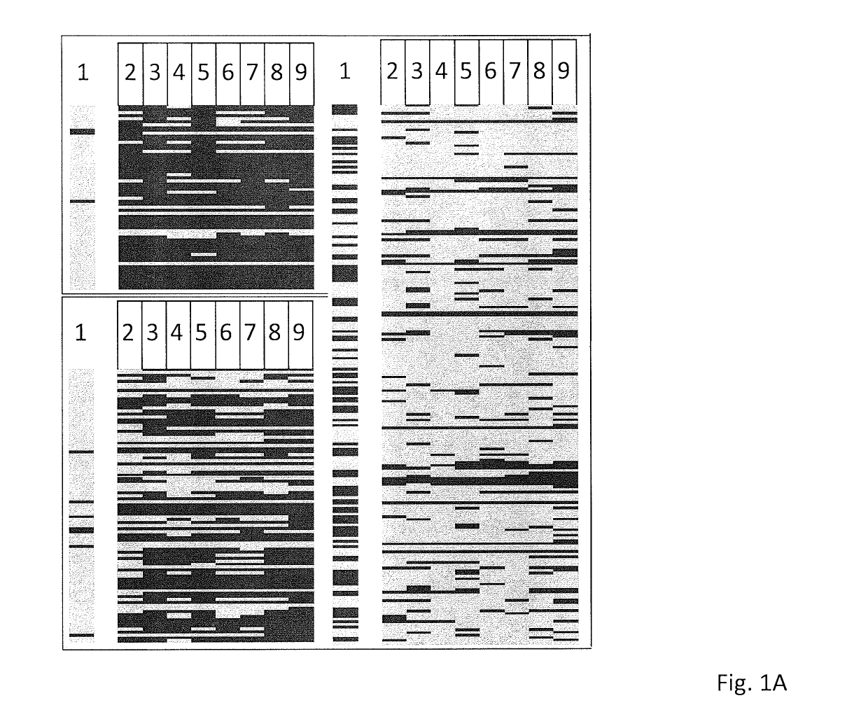

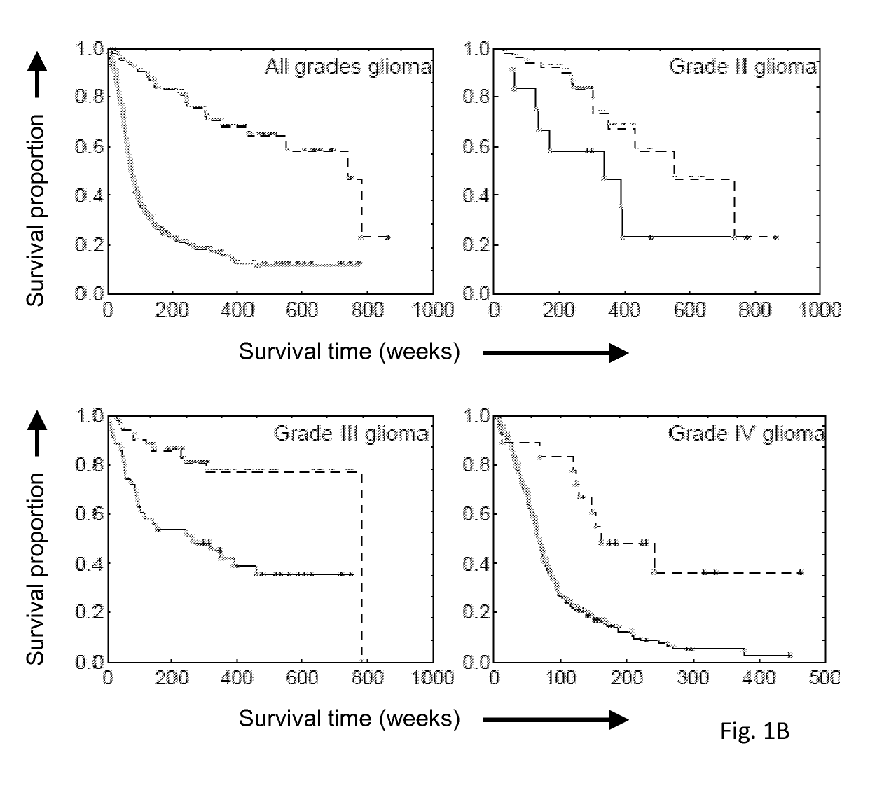

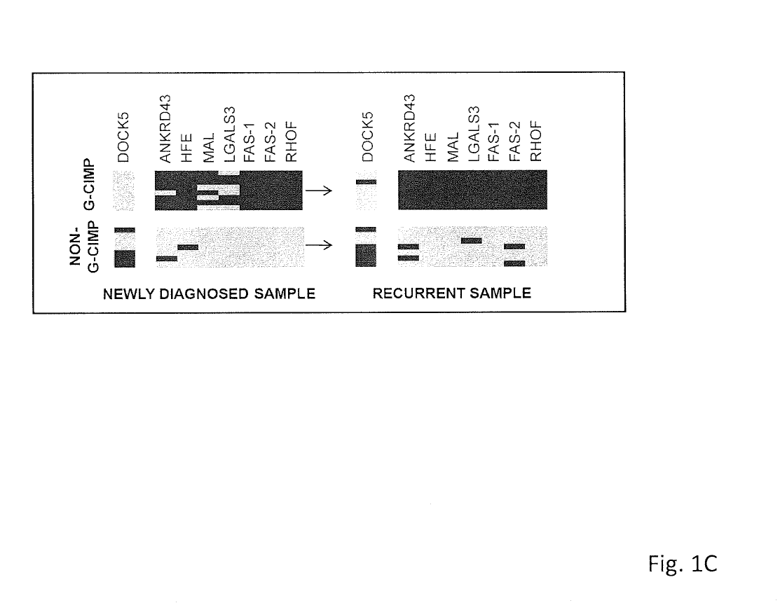

Validation of methylation biomarkers in GBM and establishment of prognostic associations in low-grade gliomas. To validate the prognostic association of methylation markers in GBM and establish its prognostic association in lower grade gliomas, MethyLight was used to assay the DNA methylation levels in eight gene regions selected from Table 1 in seven hypermethylated loci (ANKRD43, HFE, MAL, LGALS3, FAS-1, FAS-2, and RHO-F) and one hypomethylated locus, DOCK5, in the tumor samples. These eight markers were evaluated in paraffin embedded tissues from 20 TCGA samples of known favorable methylation phenotype status. All samples were scored using a requirement of DNA methylation in all seven markers of DNA hypermethylation in order to be deemed having a favorable methylation phenotype. Using these criteria, an independent set of non-TCGA GBM samples for methylation biomarker status was tested. Sixteen of 208 tumors (7.6%) were found to have a favorable methylation phenotype (FIG. 1A), very similar to the findings in TCGA data.

Based on the association of methylation marker status with features of the progressive, rather than the de novo GBM pathway, the inventors hypothesized that the favorable methylation phenotype was more common in the low- and intermediate-grade gliomas. The inventors extended this analysis by evaluating 60 grade II and 92 grade III gliomas for the 9 methylation markers. Methylation marker status correlated with improved patient survival within each WHO-recognized grade of diffuse glioma, indicating that the marker status was prognostic for glioma patient survival (p<0.032, FIG. 1B). Methylation marker status was an independent predictor (p<0.01) of survival after adjustment for patient age and tumor grade (Table 2). Together, these findings show that methylation marker status and confers improved survival in gliomas of WHO grades II, III, IV.

TABLE 2
Methylation marker status as an independent predictor of survival
after correction for patient age and tumor grade

| | Hazard ratio | p-value |
|---|---|---|
| Tumor grade | 2.1 | <0.000001 |
| Patient Age (in decades) | 1.2 | 0.000026 |
| Methylation marker status | 0.4 | 0.000020 |
| (favorable phenotype positive) | | |

These nine markers were selected based on technical advantages and expediency relating to the type of tissue specimens to be studied (archival pathology specimens which are formalin-fixed and paraffin embedded). The process of formalin fixation and paraffin embedding results in some loss in the quality of the DNA such that only a subset of markers can actually be reliably assayed, based on physical properties of PCR as well as modifications to the DNA by the formalin fixation process. Practical issues aside, one can imagine that if someone else were to base their assay on tissues of higher quality (for example frozen tissues), then another set could be chosen from the list of 200 which could essentially capture the same overall process. Alternatively, if improvements in the isolation of DNA from paraffin samples are made, then one might do a similar thing.

MethyLight technology. Tumor samples were reviewed by a neuropathologist (K.A.) to ensure accuracy of diagnosis as well as quality control to minimize normal tissue contamination. MethyLight real-time PCR strategy was performed as described previously (Eads et al., 2000; Eads et al., 1999).

Sections were cut, deparaffinized and DNA was isolated using a commercially available kit (Epicentre, Madison, Wis.). Samples were converted with bisulfite (using a kit from Zymo Research, Orange, Calif.), and then amplified by the fluorescence-based, MethyLight real-time PCR strategy as described previously (Eads et al., 2000; Eads et al., 1999). Primers, probes, amplicon sequences of the nine methylation biomarkers are shown in Table 3. Primers were tested on commercially available methylated and un methylated DNA converted with bisulfite to assure PCR specificity. To increase sensitivity, a pre-amplification step of 10 cycles was performed prior to real-time PCR. The DNA methylation levels of each gene and sample was determined by calculating delta Ct values of each methylation marker gene to the COL2A1 reference gene using ABI 7900 Sequence Detection System (Perkin-Elmer, Foster City, Calif.) or a Bio-Rad Chromo 4 Continuous Fluorescence Detector. COL2A1 serves as a reference marker to ensure the data quality. The fluorescent quencher used for the reaction is as shown in Table 3.

TABLE 3
List of primers and probes Primer and probe sequences for 9 methylation markers.

| Target | Forward Primer | Reverse Primer | Probe Sequence | Quencher | Amplicon Sequence |
|---|---|---|---|---|---|
| ANKRD | TCGTCGGTATCGA | CGATACTAAAC | AATACGCAACTC | BHQ-1 | TAGTTTCGGGGATACGTTCGGTTGGTCGCGG |
| 43 | GTAGCGG | TTCCTACAAAA | CGAACTACTAAA | | GGAAGGTATTAGGTGAGCGCGGTCGCGTTTT |
| | (SEQ ID | ACACGAC (SEQ | CCGCTTC (SEQ | | TCGGAATTTCGTTTTCGCGCGTTTCGCGGCG |
| | NO: 1) | ID NO: 2) | ID NO: 3) | | ACGCGGCGTTTATTC (SEQ ID NO: 4) |
| HFE | TTTTTGATGTTTT | CGCGCCCCTAA | CGAACTCACGCA | BHQ-1 | TTTTTGATGTTTTTGTAGATCGCGGTTTTGT |
| | TGTAGATCGCG | TTCGC (SEQ | ACAAACGCCCCT | | AGGGGCGTTTGTTGCGTGAGTTCGAGGGTTG |
| | (SEQ ID NO: 5) | ID NO: 6) | A (SEQ ID | | CGGGCGAATTAGGGGCGCG |
| | | | NO: 7) | | (SEQ ID NO: 8) |
| MAL | GTTCGGTGTAGG | ATCTACAATAA | CGACCGCCGACC | BHQ-1 | TTCGAGAGGTGTTTTGATGAGAAGGTTTGGG |
| | ATTTTAGCGTC | AAAATAAAACC | CCTTCCG | | GTTTCGGTTATTGATGGTTATTATTTTTACG |
| | (SEQ ID | GACCG (SEQ | (SEQ ID | | AGATGTTGGTTATTTACGAAGGGAGAAAGGT |
| | NO: 9) | ID NO: 10) | NO: 11) | | ACGAGGAGCGTTTGATTA |
| | | | | | (SEQ ID NO: 12) |
| DOCK5 | CGGTTCGCGGAG | AACTACTACAA | CAAACGCTTCCG | BHQ-1 | CGGTTCGCGGAGTTTAGCGAAGTTTGGCGGA |
| | TTTAGC | CTCCTCGAACT | CCATATTCCGCC | | ATATGGCGGAAGCGTTTGGGGTACGTAGGAG |
| | (SEQ ID | CCG (SEQ | (SEQ ID | | CGCGGGGCGGCGGCGGTCGGAGTTCGAGGAG |
| | NO: 13) | ID NO: 14) | NO: 15) | | TTGTAGTAGTT (SEQ ID NO: 16) |
| LGALS3 | GCGGAGTTTCGT | AATAACCAAAC | CCGCAAAACGCA | MGBNFQ | GCGGAGTTTCGTGGGTTTCGTCGTCGTCGTA |
| | GGGTTTCG | TACGACTCGTC | AACGACGAAAAT | | TTTTCGTCGTTTGCGTTTTGCGGTTTTAGAG |
| | (SEQ ID | ACC (SEQ | ACGACG (SEQ | | TAAGTTTTATTCGGTGACGAGTCGTAGTTTG |
| | NO: 17) | ID NO: 18) | ID NO: 19) | | GTTATT (SEQ ID NO: 20) |
| FAS-1 | AGGAACGTTTCG | CAACTTAACCT | TGTGTAACGAATT | MGBNFQ | AGGAACGTTTCGGGATAGGAATGTTTATTTG |
| | GGATAGGAA | ACGCGCGAAT | TTG (SEQ | | TGTAACGAATTTTGATTTTTTTTTTATTTTG |
| | (SEQ ID | (SEQ ID | ID NO: 23) | | ATTTTTTTTTTTTTTTATTCGCGCGTAGGTT |
| | NO: 21) | NO: 22) | | | AAGTTG (SEQ ID NO: 24) |
| FAS-2 | GGGTAGGAGGTC | TTCGTTACACA | TGAGTATGTTAGT | MGBNFQ | GGGTAGGAGGTCGGTTTTCGAGGTTTTTATT |
| | GGTTTTCG | AATAAACATTC | TATTGTAGGAAC | | TGAAGTGAGTATGTTAGTTATTGTAGGAACG |
| | (SEQ ID | CTATCC (SEQ | (SEQ ID | | TTTCGGGATAGGAATGTTTATTTGTGTAACG |
| | NO: 25) | ID NO: 26) | NO: 27) | | AA (SEQ ID NO: 28) |
| RHOF | GTCGTAGTCGTCG | GCTACGAACTC | AAACCCTAACCC | MGBNFQ | GTCGTAGTCGTCGTCGTTTACGATTACGATT |
| | TCGTTTACG | CGAACAATAAA | AAACCGCCGCCC | | TTTAGTTTTTTTTTGTTCGGATCGGGGGCGG |
| | (SEQ ID | TACC (SEQ | (SEQ ID | | CGGTTTGGGTTAGGGTTTCGGGGGTATTTAT |
| | NO: 29) | ID NO: 30) | NO: 31) | | TGTTCGGAGTTCGTAGT |
| | | | | | (SEQ ID NO: 32) |
| WWTR1 | TTATTACGTTTCG | CGCCCAAATAA | CGCGCTCATCCG | MGBNFQ | GGGTAAGAGGAGACGGGTGTTTTTTATTTAT |
| | ATTTCGGAAGTTC | TACCCGCTAAA | ACACCACTCCAA | | TTTTTTCGGTCGCGCGGATTTTTTTCGTTTA |
| | G (SEQ | AC (SEQ | (SEQ ID | | GATTTGTATTTGTATTTTTTTGAGTTTATTA |
| | ID NO: 33) | ID NO: 34) | NO: 35) | | CGGATTTGGGGCGGGATT |
| | | | | | (SEQ ID NO: 36) |
| COL2A1 | TCTAACAATTATA | GGGAAGATGGG | CCTTCATTCTAAC | TAMRA | |
| | AACTCCAACCAC | ATAGAAGGGAA | CCAATACCTATCC | | |
| | CAA (SEQ | TAT (SEQ | CACCTCTAAA | | |
| | ID NO: 37) | ID NO: 38) | (SEQ ID | | |
| | | | NO: 39) | | |

Multivariate analysis (Table 4) shows that methylation status is an independent predictor of outcome after adjusting for relevant clinical variables. After adjustment for patient age and tumor grade methylation status was highly significant. The hazard ratio (HR) of 0.34 for methylation status shows that it is a favorable prognostic marker.

TABLE 4
Multivariate analysis

| Variable | HR (hazard ratio) | p-value |
|---|---|---|
| Tumor Grade | 1.87 | 0.0000060 |
| Patient age | 1.02 | 0.0000034 |
| Methylation status | 0.34 | 0.0000003 |

Methylation biomarkers predict outcome in patients with glioma as well as an association of methylation with tumor grade. As shown in FIG. 1A, tumor samples from 355 patients with diffuse glioma were tested for a methylation biomarker panel. Tumors were considered heavily methylated if 7 of 9 markers were positive. Patients were divided into two groups based on heavy methylation (dashed line) vs. light methylation (solid line). On the left all patients are shown. The data indicates significantly improved survival for patients whose tumors show heavy methylation. The remaining 3 panels show methylation status vs. survival in patients of all 3 glioma grades indicating that this biomarker panel is predictive of outcome in all grades of glioma. Inspection of the data when stratified by grade shows an increase in favorable prognostic markers in grade II tumor (upper left), an intermediate amount in grade III tumors (lower left), and a low amount in grade IV tumors (right).

Stability of methylation marker status at recurrence. Since epigenetic events can be dynamic processes, the inventors examined whether methylation marker status was a stable event in glioma or whether it was subject to change over the course of the disease. To test this, the inventors obtained a set of samples from 15 patients who received a second surgical procedure following tumor recurrence, with time intervals of up to eight years between initial and second surgical procedures. The inventors used the eight-gene MethyLight panel to determine their methylation marker status and found that eight samples were had a favorable methylation profile, while seven had an unfavorable methylation profile. Interestingly, among the methylation-favorable cases, 8/8 (100%) recurrent samples retained positive profile. Similarly, among seven methylation-unfavorable cases, all seven remained negative for the methylation profile upon recurrence, indicating stability of the methylation phenotype over time (FIG. 1C).

In summary, these data indicate that methylation marker status stratify gliomas into two distinct subgroups with different molecular and clinical phenotypes. These molecular classifications have implications for differential therapeutic strategies for glioma patients. Further observation and characterization of molecular subsets of glioma will likely provide additional information enabling insights into the development and progression of glioma, and may lead to targeted drug treatment for patients with these tumors.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,786,146
U.S. Pat. No. 5,972,602
U.S. Pat. No. 6,033,854
U.S. Pat. No. 6,180,349
U.S. Publn. 2004/0132048
U.S. Publn. 2005/0069879
U.S. Ser. No. 10/971,339
U.S. Ser. No. 10/971,986
U.S. Ser. No. 11/071,013
Adamson et al., Expert Opin. Investig. Drugs, 18:1061-1083, 2009.
Akerstrom et al., J. Immunol., 135:2589-2542, 1985.
Amundadottir et al., Nat. Genet., 38, 652-658, 2006.
Ausubel et al., In: Current Protocols in Molecular Biology, 1995.
Ausubel, In: Current Protocols in Molecular Biology, 1987-2006.
Balss et al., Acta Neuropathol., 116, 597-602, 2008.
Barringer et al., Gene, 89:117, 1990.
Basic and Clinical Immunology, Stites & Terr (Ed.), 7^{th} Ed., 1991.
Bibikova et al., Genome Res., 16, 383-393, 2006.
Bleeker et al., Hum. Mutat., 30:7-11, 2009.
Brandes et al., Ann. Oncol., 12:255-257, 2001.
Campan et al., Methods Mol. Biol., 507:325-337, 2009.
Canoll and Goldman, Acta Neuropathol., 116:465-477, 2008.
Coligan, In: Current Protocols in Immunology, 1991.
Colman et al., Neuro-Oncology, doi:10.1093/neuonc/nop007, 2009.
Dang et al., Nature, 10.1038/nature08617, 2009.
Davies and Enns, J. Biolog. Chem., 279(24):25085-25092, 2004.
DeGraves et al., Biotechniques, 34(1):106-10, 112-5, 2003.
Deiman et al., Mol. Biotechnol., 20(2):163-79, 2002.
Dennis et al., Genome Biol., 4:P3, 2003.
Dirks, Nature, 444:687-688, 2006.
Ducray et al., Mol. Cancer, 7:41, 2008.
Eads et al., Cancer Res., 59, 2302-2306, 1999.
Eads et al., Nucleic Acids. Res., 28:E32, 2000.
Enzyme Immunoassay, Maggio (Ed.), 1980.
Freedman et al., Proc. Natl. Acad. Sci. USA, 103:14068-14073, 2006.
Frigola et al., Nat. Genet., 38:540-549, 2006.
Frommer et al., Proc. Natl. Acad. Sci. USA, 89:1827-1831, 1992.
Furnari et al., Genes Dev., 21, 2683-2710, 2007.
Galli et al., Cancer Res., 64:7011-7021, 2004.
Gentleman et al., Genome Biol., 5:R80, 2004.
Gibson et al., Genome Research 6:995-1001 (1996
Gillies and Lorimer, Cell Cycle, 6:2005-2009, 2007.
Goding, In: Monoclonal Antibodies: Principles and Practice, 2^{nd} Ed., 1986.
Gonzalgo & Jones, Nucleic Acids Res., 25:2529-2531, 1997.
Graff et al., J. Biol. Chem., 272:22322-22329, 1997.
Guatelli et al., Proc. Nat. Acad. Sci. USA, 87:1874, 1990.
Haiman et al., Nat. Genet., 39:638-644, 2007b.
Haiman et al., Nat. Genet., 39:954-956, 2007a.
Harlow & Lane, Antibodies: A Laboratory Manual, 1988.
Harlow & Lane, Antibodies: A Laboratory Manual, 1999.
Harrison's Principles Internal Medicine, Kaspar et al. (Eds), 16^{th} Ed., McGraw-Hill, Inc., 2005.
Hartmann et al., Acta Neuropathol., 118(4):469-74, 2009.
Herman et al., Proc. Nat. Acad. Sci. USA, 93:9821-9826, 1996.
Huang et al., Acta Neuropathol., 118:469-474, 2009.
Huse et al., Science, 246:1275-1281, 1989.
Innis et al., In: PCR Protocols, A Guide to Methods and Applications, Academic Press, Inc. NY, 1991.
Kirov et al., Genomics, 82:433-440, 2003.
Kohler & Milstein, Nature, 256:495-497, 1975.
Kronval et al., J. Immunol., 111:1401-1406, 1973.
Kwoh et al., Proc. Natl. Acad. Sci. USA, 86:1173, 1989.
Landegren et al., Science, 241:1077, 1988.
Liang et al., Proc. Natl. Acad. Sci. USA, 86102:5814-5819, 2005.
Liu et al., Cancer Genomics Proteomics, 6:131-139, 2009.
Madhavan et al., Mol. Cancer Res., 7:157-167, 2009.
Martinez et al., J Neurooncol., 10.1007/s11060-009-9967-4, 2009b.
Momparler, Oncogene, 22:6479-83, 2003.
Monroe et al., Amer. Clin. Prod. Rev., 5:34-41, 1986.
Monti et al., Machine Learning, 52:91-118, 2003.
Olek et al. Nat. Genet., 17(3):275-6, 1997.
Parsons et al., Science, 321:1807-1812, 2008.
PCT Publn. WO 00/70090
Phillips et al., Cancer Cell, 9:157-173, 2006.
Reich et al., Nat. Genet., 38:500-501, 2006.
Rein, et al. Nucleic Acids Res., 26 (10):2255-64, 1998.
Sadri & Hornsby, Nucl. Acids Res., 24:5058-5059, 1996.
Sambrook and Russell, Molecular Cloning: A Laboratory Manual, 3^{rd} Ed. Cold Spring Harbor Lab. Press, 2000.
Sambrook and Russell, Molecular Cloning: A Laboratory Manual, 3^{rd} Ed. Cold Spring Harbor Lab. Press, 2001.
Sanson et al., J. Clin. Oncol., 27:4150-4154, 2009.
Schaefer et al., Int. J. Cancer, 126(5):1166-76, 2009.
Schumacher et al., Cancer Res., 67:2951-2956, 2007.
Shete et al., Nat. Genet., 41:899-904, 2009.
Subramanian et al., Proc. Natl. Acad. Sci. USA, 102:15545-15550, 2005.
Sun et al., Cancer Cell, 9:287-300, 2006.
Sung et al., J. Biol. Chem., 284:21941-21954, 2009.
Taylor et al., PLoS One, 3:e3179, 2008.
The Cancer Genome Atlas Research Network, Nature, 455:1061-1068, 2008.
Toyota et al., Cancer Res., 59:2307-12, 1999.
Visakorpi et al., Cancer Res., 55:342-347, 1995.
Ward et al., Nature, 341:544-546, 1989.
Weisenberger et al., Nat. Genet., 38:787-793, 2006.
Wu and Wallace, Genomics, 4:560, 1989.
Xiong & Laird, Nucleic Acids Res., 25:2532-2534, 1997.
Yan et al., N Engl. J. Med., 360:765-773, 2009.
Yeager et al., Nat. Genet., 39:645-649, 2007.

What is claimed is:

1. A method for determining whether a subject's brain tumor has a favorable methylation phenotype, the method comprising determining whether the subject's brain tumor has a methylation status of seven or more of methylation markers selected from the group consisting of: hyper-methylation of ANKRD43 (ankyrin repeat domain 43) gene, hyper-methylation of HFE (human hemochromatosis protein) gene, hyper-methylation of MAL (T cell differentiation protein) gene, hyper-methylation of LGALS3 (galectin-3) gene, hyper-methylation of FAS-1 marker, hyper-methylation of FAS-2 marker; hyper-methylation of RHO-F (ras homolog gene family, member F) gene, hyper-methylation of WWTR1 (WW domain containing transcription regulator 1) gene, and hypo-methylation of DOCK5 (dedicator of cytokinesis 5) gene, the existence of such a methylation status being indicative of a favorable methylation phenotype, wherein:
i) if the subject's cancer has the favorable methylation phenotype, the subject is more likely to exhibit a favorable prognosis; and/or
ii) if the subject's cancer does not have the favorable methylation phenotype, the subject is less likely to exhibit a favorable prognosis.

2. The method of claim 1, wherein the cancer is a primary brain tumor.

3. The method of claim 1, wherein the cancer is secondary brain tumor.

4. The method of claim 1, wherein the cancer is a glioma.

5. The method of claim 4, wherein the cancer is glioblastoma.

6. The method of claim 5, wherein the cancer is secondary glioblastoma.

7. The method of claim 1, wherein the subject has or is suspected to have a recurrent brain tumor.

8. The method of claim 1, wherein the method comprises determining a methylation status of one or more non-coding regions.

9. The method of claim 8, wherein the one or more non-coding regions comprise at least one promoter.

10. The method of claim 1, wherein the method comprises obtaining a sample of the subject.

11. The method of claim 10, wherein the sample is a preserved sample.

12. The method of claim 11, wherein the preserved sample is a formalin-fixed, paraffin-embedded (FFPE) sample.

13. The method of claim 1, wherein the method comprises isolating nucleic acids of the subject's cancer.

14. The method of claim 1, wherein the method comprises assaying nucleic acids of the subject's cancer.

15. The method of claim 14, wherein assaying nucleic acids of the subject comprises a methylation assay.

16. The method of claim 15, wherein the methylation assay comprises Southern blotting, single nucleotide primer extension (SNuPE), methylation-specific PCR (MSPCR), restriction landmark genomic scanning for methylation (RLGS-M), HpaII-tiny fragment enrichment by ligation-mediated PCR (HELP assay), CpG island microarray, ChIP-chip (chromatin immnuprecipitation-on-chip), ChIP-seq (chromatin immunoprecipitation-sequencing), methylated DNA immunoprecipitation (MeDIP), bisulfite sequencing, combined bisulfite restriction analysis (COBRA) or a microarray-based methylation profiling.

17. The method of claim 16, wherein the microarray-based methylation profiling is Infinium® methylation assay or GoldenGate® methylation assay.

18. The method of claim 1, wherein the method comprises analyzing a predetermined methylation profile of the subject's cancer.

19. The method of claim 1, further comprising recording the methylation phenotype determination in a tangible medium.

20. The method of claim 1, further comprising reporting the methylation phenotype determination to the subject, a health care payer, a physician, an insurance agent, or an electronic system.

21. The method of claim 1, wherein the favorable prognosis comprises a higher chance of survival as compared with a reference level.

22. A method for treating a patient having a brain tumor comprising administering one or more conventional cancer treatment to a patient determined to have a favorable methylation phenotype, wherein a favorable phenotype is defined as having a brain tumor that comprises seven or more of methylation markers selected from the group consisting of: hyper-methylation of ANKRD43 (ankyrin repeat domain 43) gene, hyper-methylation of HFE (human hemochromatosis protein) gene, hyper-methylation of MAL (T cell differentiation protein) gene, hyper-methylation of LGALS3 (galectin-3) gene, hyper-methylation of FAS-1 marker, hyper-methylation of FAS-2 marker; hyper-methylation of RHO-F (ras homolog gene family, member F) gene, hyper-methylation of WWTR1 (WW domain containing transcription regulator 1) gene, and hypo-methylation of DOCK5 (dedicator of cytokinesis 5) gene.

23. The method of claim 22, wherein one or more conventional cancer treatments comprise chemotherapy, radiation therapy, and/or surgery.

24. A method for treating a patient having a brain tumor comprising administering one or more alternative cancer treatments to a patient determined not to have a favorable methylation phenotype, wherein a favorable phenotype is defined as having a brain tumor that comprises seven or more of methylation markers selected from the group consisting of: hyper-methylation of ANKRD43 (ankyrin repeat domain 43) gene, hyper-methylation of HFE (human hemochromatosis protein) gene, hyper-methylation of MAL (T cell differentiation protein) gene, hyper-methylation of LGALS3 (galectin-3) gene, hyper-methylation of FAS-1 marker, hyper-methylation of FAS-2 marker; hyper-methylation of RHO-F (ras homolog gene family, member F) gene, hyper-methylation of WWTR1 (WW domain containing transcription regulator 1) gene, and hypo-methylation of DOCK5 (dedicator of cytokinesis 5) gene.

25. The method of claim 24, wherein the one or more alternative cancer treatments comprise angiogenesis inhibitor therapy, immunotherapy, gene therapy, hyperthermia, photodynamic therapy, and/or targeted cancer therapy.

26. A kit comprising a plurality of primers or probes specific for determining a methylation status of seven or more of methylation markers in Table 1.

27. A tangible, computer-readable medium comprising a methylation profile of a subject having a brain tumor, wherein the methylation profile comprises a methylation status of seven or more of methylation markers in Table 1.


SEQUENCE LISTING (39 sequences)

SEQ ID NO: 1 - DNA, 20 bp, Artificial Sequence
  Synthetic primer
tcgtcggtatcgagtagcgg

SEQ ID NO: 2 - DNA, 29 bp, Artificial Sequence
  Synthetic primer
cgatactaaacttcctacaaaaacacgac

SEQ ID NO: 3 - DNA, 31 bp, Artificial Sequence
  Synthetic primer
aatacgcaactccgaactactaaaccgcttc

SEQ ID NO: 4 - DNA, 108 bp, Artificial Sequence
  Synthetic primer
tagtttcggggatacgttcggttggtcgcggggaaggtattaggtgagcgcggtcgcgtttttcggaatttcgttttcgcgcgtttcgcggcgacgcggcgtttattc

SEQ ID NO: 5 - DNA, 24 bp, Artificial Sequence
  Synthetic primer
tttttgatgtttttgtagatcgcg

SEQ ID NO: 6 - DNA, 16 bp, Artificial Sequence
  Synthetic primer
cgcgcccctaattcgc

SEQ ID NO: 7 - DNA, 25 bp, Artificial Sequence
  Synthetic primer
cgaactcacgcaacaaacgccccta

SEQ ID NO: 8 - DNA, 81 bp, Artificial Sequence
  Synthetic primer
tttttgatgtttttgtagatcgcggttttgtaggggcgtttgttgcgtgagttcgagggttgcgggcgaattaggggcgcg

SEQ ID NO: 9 - DNA, 23 bp, Artificial Sequence
  Synthetic primer
gttcggtgtaggattttagcgtc

SEQ ID NO: 10 - DNA, 27 bp, Artificial Sequence
  Synthetic primer
atctacaataaaaaataaaaccgaccg

SEQ ID NO: 11 - DNA, 19 bp, Artificial Sequence
  Synthetic primer
cgaccgccgaccccttccg

SEQ ID NO: 12 - DNA, 111 bp, Artificial Sequence
  Synthetic primer
ttcgagaggtgttttgatgagaaggtttggggtttcggttattgatggttattatttttacgagatgttggttatttacgaagggagaaaggtacgaggagcgtttgatta

SEQ ID NO: 13 - DNA, 18 bp, Artificial Sequence
  Synthetic primer
cggttcgcggagtttagc

SEQ ID NO: 14 - DNA, 25 bp, Artificial Sequence
  Synthetic primer
aactactacaactcctcgaactccg

SEQ ID NO: 15 - DNA, 24 bp, Artificial Sequence
  Synthetic primer
caaacgcttccgccatattccgcc

SEQ ID NO: 16 - DNA, 104 bp, Artificial Sequence
  Synthetic primer
cggttcgcggagtttagcgaagtttggcggaatatggcggaagcgtttggggtacgtaggagcgcggggcggcggcggtcggagttcgaggagttgtagtagtt

SEQ ID NO: 17 - DNA, 20 bp, Artificial Sequence
  Synthetic primer
gcggagtttcgtgggtttcg

SEQ ID NO: 18 - DNA, 25 bp, Artificial Sequence
  Synthetic primer
aataaccaaactacgactcgtcacc

SEQ ID NO: 19 - DNA, 30 bp, Artificial Sequence
  Synthetic primer
ccgcaaaacgcaaacgacgaaaatacgacg

SEQ ID NO: 20 - DNA, 99 bp, Artificial Sequence
  Synthetic primer
gcggagtttcgtgggtttcgtcgtcgtcgtattttcgtcgtttgcgttttgcggttttagagtaagttttattcggtgacgagtcgtagtttggttatt

SEQ ID NO: 21 - DNA, 21 bp, Artificial Sequence
  Synthetic primer
aggaacgtttcgggataggaa

SEQ ID NO: 22 - DNA, 21 bp, Artificial Sequence
  Synthetic primer
caacttaacctacgcgcgaat

SEQ ID NO: 23 - DNA, 16 bp, Artificial Sequence
  Synthetic primer
tgtgtaacgaattttg

SEQ ID NO: 24 - DNA, 99 bp, Artificial Sequence
  Synthetic primer
aggaacgtttcgggataggaatgtttatttgtgtaacgaattttgattttttttttattttgatttttttttttttttattcgcgcgtaggttaagttg

SEQ ID NO: 25 - DNA, 20 bp, Artificial Sequence
  Synthetic primer
gggtaggaggtcggttttcg

SEQ ID NO: 26 - DNA, 28 bp, Artificial Sequence
  Synthetic primer
ttcgttacacaaataaacattcctatcc

SEQ ID NO: 27 - DNA, 25 bp, Artificial Sequence
  Synthetic primer
tgagtatgttagttattgtaggaac

SEQ ID NO: 28 - DNA, 95 bp, Artificial Sequence
  Synthetic primer
gggtaggaggtcggttttcgaggtttttatttgaagtgagtatgttagttattgtaggaacgtttcgggataggaatgtttatttgtgtaacgaa

SEQ ID NO: 29 - DNA, 22 bp, Artificial Sequence
  Synthetic primer
gtcgtagtcgtcgtcgtttacg

SEQ ID NO: 30 - DNA, 26 bp, Artificial Sequence
  Synthetic primer
gctacgaactccgaacaataaatacc

SEQ ID NO: 31 - DNA, 24 bp, Artificial Sequence
  Synthetic primer
aaaccctaacccaaaccgccgccc

SEQ ID NO: 32 - DNA, 110 bp, Artificial Sequence
  Synthetic primer
gtcgtagtcgtcgtcgtttacgattacgatttttagtttttttttgttcggatcgggggcggcggtttgggttagggtttcgggggtatttattgttcggagttcgtagt

SEQ ID NO: 33 - DNA, 27 bp, Artificial Sequence
  Synthetic primer
ttattacgtttcgatttcggaagttcg

SEQ ID NO: 34 - DNA, 24 bp, Artificial Sequence
  Synthetic primer
cgcccaaataatacccgctaaaac

SEQ ID NO: 35 - DNA, 24 bp, Artificial Sequence
  Synthetic primer
cgcgctcatccgacaccactccaa

SEQ ID NO: 36 - DNA, 111 bp, Artificial Sequence
  Synthetic primer
gggtaagaggagacgggtgttttttatttatttttttcggtcgcgcggatttttttcgtttagatttgtatttgtatttttttgagtttattacggatttggggcgggatt

SEQ ID NO: 37 - DNA, 28 bp, Artificial Sequence
  Synthetic primer
tctaacaattataaactccaaccaccaa

SEQ ID NO: 38 - DNA, 25 bp, Artificial Sequence
  Synthetic primer
gggaagatgggatagaagggaatat

SEQ ID NO: 39 - DNA, 36 bp, Artificial Sequence
  Synthetic primer
ccttcattctaacccaatacctatcccacctctaaa